(19) United States
(12) Patent Application Publication
(10) Pub. No.: US 2026/0066069 A1
(43) Pub. Date: Mar. 5, 2026

(54) INFORMATION PROCESSING APPARATUS, INFORMATION PROCESSING METHOD, AND NON-TRANSITORY COMPUTER READABLE MEDIUM

(71) Applicant: NEC Corporation, Tokyo (JP)

(72) Inventors: Yuki Kusano, Tokyo (JP); Shohei Yamaguchi, Tokyo (JP); Takayuki Yamamoto, Tokyo (JP); Kosuke Osa, Tokyo (JP)

(73) Assignee: NEC Corporation, Tokyo (JP)

(21) Appl. No.: 19/307,172

(22) Filed: Aug. 22, 2025

(30) Foreign Application Priority Data
Aug. 28, 2024 (JP) 2024-146601

(51) Int. Cl. G16H 10/60

(52) U.S. Cl. CPC G16H 10/60

(57) ABSTRACT

An information processing apparatus includes an acquisition unit that obtains a written request for requesting a change in a specification of an electronic medical record, and a generation unit that generates a review result of the written request by inputting the written request and a prompt to a language model that has been subjected to machine learning, in which the generation unit generates the review result of at least one of the review regarding a patient information handover or the review regarding suppression of falsification of information associated with the electronic medical record to support decision making of a user.

### INCORPORATION BY REFERENCE

This application is based upon and claims the benefit of priority from Japanese patent application No. 2024-146601, filed on Aug. 28, 2024, the disclosure of which is incorporated herein in its entirety by reference.

### TECHNICAL FIELD

The present disclosure relates to an information processing apparatus, an information processing method, and an information processing program.

### BACKGROUND ART

Techniques for supporting creation of documents have been known. For example, PTL 1 discloses an examination business document creation support device that supports creation of an examination business document in a financial institution.

PTL 1: JP 7396582 B1

### SUMMARY

Examples of documents other than the examination business document described above include a written request for requesting a change in specifications of an electronic medical record. The specifications of the electronic medical record may be changed in response to a request from medical personnel who use the electronic medical record. In that case, an inappropriate change in the specifications of the electronic medical record may lead to a medical accident. Thus, there is a need for a technique for supporting creation of a written request for requesting a change in specifications of an electronic medical record.

The present disclosure has been conceived in view of the problem described above, and an example object thereof is to provide a technique for supporting creation of a written request for requesting a change in specifications of an electronic medical record.

An information processing apparatus according to an example aspect of the present disclosure includes an acquisition means for obtaining a written request for requesting a change in a specification of an electronic medical record, and a generation means for generating a review result of the written request by inputting the written request and a prompt for instructing execution of a review of the written request to a language model that has been subjected to machine learning, in which the generation means generates the review result of at least one of the review regarding a patient information handover or the review regarding suppression of falsification of information associated with the electronic medical record.

An information processing method according to an example aspect of the present disclosure includes acquisition processing in which at least one processor obtains a written request for requesting a change in a specification of an electronic medical record, and generation processing in which the at least one processor generates a review result of the written request by inputting the written request and a prompt for instructing execution of a review of the written request to a language model that has been subjected to machine learning, in which the at least one processor generates, in the generation processing, the review result of at least one of the review regarding a patient information handover or the review regarding suppression of falsification of information associated with the electronic medical record.

An information processing program according to an example aspect of the present disclosure is a program that causes a computer to function as an information processing apparatus, and causes the computer to function as an acquisition means for obtaining a written request for requesting a change in a specification of an electronic medical record, and a generation means for generating a review result of the written request by inputting the written request and a prompt for instructing execution of a review of the written request to a language model that has been subjected to machine learning, in which the generation means generates the review result of at least one of the review regarding a patient information handover or the review regarding suppression of falsification of information associated with the electronic medical record.

According to an example aspect of the present disclosure, an exemplary effect is exerted in which a technique for supporting creation of a written request for requesting a change in specifications of an electronic medical record may be provided.

### BRIEF DESCRIPTION OF DRAWINGS

The above-described and other aspects, features, and advantages of the present disclosure will become more apparent from the following descriptions of specific illustrative example embodiments along with the accompanying drawings.

FIG. 1 is a block diagram illustrating a configuration of an information processing apparatus according to the present disclosure;

FIG. 2 is a flowchart illustrating a flow of an information processing method according to the present disclosure;

FIG. 3 is a diagram illustrating an outline of an information processing system using the information processing apparatus according to the present disclosure;

FIG. 4 is a block diagram illustrating a configuration of the information processing apparatus according to the present disclosure;

FIG. 5 is a flowchart illustrating a flow of the information processing method according to the present disclosure; and

FIG. 6 is a block diagram illustrating a configuration of a computer that functions as the information processing apparatus according to the present disclosure.

### EXAMPLE EMBODIMENT

Hereinafter, example embodiments of the present disclosure will be exemplified. However, the present disclosure is not limited to the following illustrative example embodiments, and various modifications may be made within the scope described in the claims. For example, example embodiments obtained by appropriately combining techniques (some or all of objects or methods) adopted in the following illustrative example embodiments may also fall within the scope of the present disclosure. Example embodiments obtained by appropriately omitting some of the techniques adopted in the following illustrative example embodiments may also fall within the scope of the present disclosure. Effects mentioned in the following illustrative example embodiments are examples of effects expected in the illustrative example embodiments, and do not define the extension of the present disclosure. That is, example embodiments that do not exert the effects mentioned in the following illustrative example embodiments may also fall within the scope of the present disclosure.

First Illustrative Example Embodiment

A first illustrative example embodiment, which is an example of the example embodiments of the present disclosure, will be described in detail with reference to the drawings. The present illustrative example embodiment is a basic form of each of the illustrative example embodiments to be described below. An application range of each technique adopted in the present illustrative example embodiment is not limited to the present illustrative example embodiment. That is, each technique adopted in the present illustrative example embodiment may also be adopted in another illustrative example embodiment included in the present disclosure as long as no particular technical problem is raised. Each technique illustrated in the drawings referred to for describing the present illustrative example embodiment may also be adopted in another illustrative example embodiment included in the present disclosure as long as no particular technical problem is raised.

### (Configuration of Information Processing Apparatus 1)

A configuration of an information processing apparatus 1 will be described with reference to FIG. 1. FIG. 1 is a block diagram illustrating the configuration of the information processing apparatus 1. As illustrated in FIG. 1, the information processing apparatus 1 includes an acquisition unit 11 and a generation unit 12. In the present illustrative example embodiment, the acquisition unit 11 and the generation unit 12 implement an acquisition means and a generation means, respectively.

### (Acquisition Unit 11)

The acquisition unit 11 obtains a written request for requesting a change in specifications of an electronic medical record. The acquisition unit 11 supplies the obtained written request to the generation unit 12.

### (Generation Unit 12)

The generation unit 12 inputs, to a language model that has been subjected to machine learning, the written request obtained by the acquisition unit 11 and a prompt for instructing execution of a review of the written request, thereby generating a review result of the written request.

The generation unit 12 further generates a review result of at least one of a review regarding a patient information handover and a review regarding suppression of falsification of electronic medical record information.

### (Effects of Information Processing Apparatus 1)

As described above, the information processing apparatus 1 employs the configuration including the acquisition unit 11 that obtains the written request for requesting a change in the specifications of the electronic medical record and the generation unit 12 that generates a review result of the written request by inputting the written request obtained by the acquisition unit 11 and the prompt for instructing the execution of the review of the written request to the language model that has been subjected to the machine learning. The generation unit 12 further generates a review result of at least one of a review regarding a patient information handover and a review regarding suppression of falsification of electronic medical record information.

In the first illustrative example embodiment, the information processing apparatus 1 generates a review result of at least one of the review regarding the patient information handover and the review regarding the suppression of the falsification of the electronic medical record information in the written request for requesting a change in the specifications of the electronic medical record. By using such a review result, it becomes possible to appropriately correct an inappropriate summary that may lead to a medical accident, whereby the information processing apparatus 1 is enabled to support creation of the written request for requesting a change in the specifications of the electronic medical record.

### (Exemplary Implementation by Program)

In a case where the information processing apparatus 1 is configured by a computer including at least one processor and a memory, the memory stores the following program. The program is a program that causes the computer to function as the information processing apparatus 1 and causes the computer to function as the acquisition unit 11 that obtains the written request for requesting a change in the specifications of the electronic medical record and the generation unit 12 that generates the review result of the written request by inputting the written request and the prompt for instructing the execution of the review of the written request to the language model that has been subjected to the machine learning, and the generation unit 12 generates the review result of at least one of the review regarding the patient information handover and the review regarding the suppression of the falsification of the electronic medical record information.

### (Flow of Information Processing Method S1)

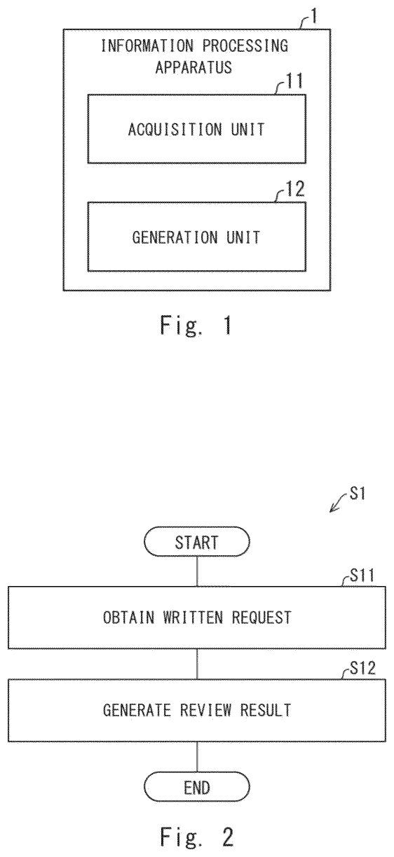

A flow of an information processing method S1 will be described with reference to FIG. 2. FIG. 2 is a flowchart illustrating the flow of the information processing method S1. As illustrated in FIG. 2, the information processing method S1 includes acquisition processing S11 and generation processing S12.

### (Acquisition Processing S11)

In the acquisition processing S11, the acquisition unit 11 obtains the written request for requesting a change in the specifications of the electronic medical record. The acquisition unit 11 supplies the obtained written request to the generation unit 12.

### (Generation Processing S12)

In the generation processing S12, the generation unit 12 inputs, to the language model that has been subjected to the machine learning, the written request obtained by the acquisition unit 11 and the prompt for instructing the execution of the review of the written request, thereby generating a review result of the written request.

In the generation processing S12, the generation unit 12 further generates a review result of at least one of the review regarding the patient information handover and the review regarding the suppression of the falsification of the electronic medical record information.

### (Effects of Information Processing Method S1)

As described above, the information processing method S1 employs the configuration including the acquisition processing S11 in which the acquisition unit 11 obtains the written request for requesting a change in the specifications of the electronic medical record and the generation processing S12 in which the generation unit 12 generates a review result of the written request by inputting the written request obtained by the acquisition unit 11 and the prompt for instructing the execution of the review of the written request to the language model that has been subjected to the machine learning. In the generation processing S12, the generation unit 12 further generates a review result of at least one of the review regarding the patient information handover and the review regarding the suppression of the falsification of the electronic medical record information.

Thus, according to the information processing method S1, effects similar to those of the information processing apparatus 1 described above may be obtained.

### Second Illustrative Example Embodiment

A second illustrative example embodiment, which is an example of the example embodiments of the present disclosure, will be described in detail with reference to the drawings. Components having the same functions as the components described in the illustrative example embodiment described above are denoted by the same reference numerals, and descriptions thereof will be omitted as appropriate. An application range of each technique adopted in the present illustrative example embodiment is not limited to the present illustrative example embodiment. That is, each technique adopted in the present illustrative example embodiment may also be adopted in another illustrative example embodiment included in the present disclosure as long as no particular technical problem is raised. Each technique illustrated in the drawings referred to for describing the present illustrative example embodiment may also be adopted in another illustrative example embodiment included in the present disclosure as long as no particular technical problem is raised.

### (Outline of Information Processing Apparatus 1A)

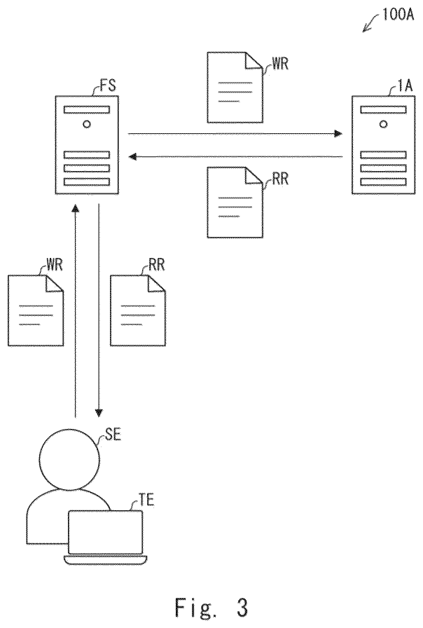

An information processing apparatus 1A is a device that reviews the written request for requesting a change in the specifications of the electronic medical record. An exemplary information processing system using the information processing apparatus 1A will be described with reference to FIG. 3. FIG. 3 is a diagram illustrating an outline of an information processing system 100A using the information processing apparatus 1A.

In the information processing system 100A, a system engineer SE creates a written request WR for requesting a change in the specifications of the electronic medical record using a terminal TE. As an example, the system engineer SE receives a request for customizing the electronic medical record from a user (medical personnel such as a doctor or a nurse) of the electronic medical record, and creates the written request WR based on the request.

Upon creation of the written request WR, the terminal TE transmits the written request WR to the file server FS. Upon acquisition of the written request WR, the file server FS stores the written request WR in a storage unit included in the file server FS.

The information processing apparatus 1A monitors whether the written request WR is stored in the storage unit included in the file server FS every predetermined period of time, such as five minutes. In a case where the written request WR is stored in the storage unit included in the file server FS, the information processing apparatus 1A obtains the written request WR. Then, the information processing apparatus 1A reviews the written request WR. The information processing apparatus 1A reviews the written request WR for each of one or a plurality of review items. The content to be reviewed by the information processing apparatus 1A is not particularly limited.

The information processing apparatus 1A transmits a review result RR, which is a result of the review, to the file server FS. Upon acquisition of the review result RR, the file server FS transmits it to the terminal TE. Then, the system engineer SE refers to the review result RR, and corrects the written request.

### (Configuration of Information Processing Apparatus 1A)

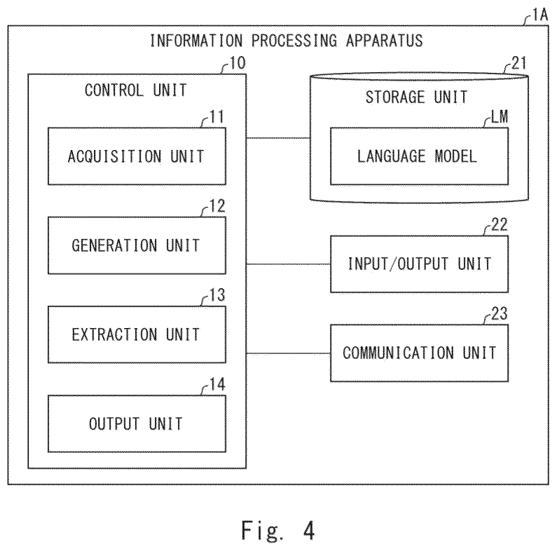

A configuration of the information processing apparatus 1A will be described with reference to FIG. 4. FIG. 4 is a block diagram illustrating the configuration of the information processing apparatus 1A. As illustrated in FIG. 4, the information processing apparatus 1A includes a control unit 10, a storage unit 21, an input/output unit 22, and a communication unit 23.

### (Storage Unit 21)

The storage unit 21 stores data to be referred to by the control unit 10. Examples of the storage unit 21 include, but are not limited to, a flash memory, a hard disk drive (HDD), a solid state drive (SSD), and a combination thereof.

Examples of the data stored in the storage unit 21 include a language model LM. The language model LM is a language model trained in such a way that the review result RR of the written request WR is output using, as an input, the written request WR and a prompt for instructing execution of a review of the written request WR. The state that the language model LM is stored in the storage unit 21 indicates that parameters defining the language model LM are stored in the storage unit 21.

The language model LM may be stored in a device different from the information processing apparatus 1A. In that case, the information processing apparatus 1A outputs, to the device, the written request WR and the prompt for instructing the execution of the review of the written request WR via the communication unit 23 to be described later. Then, the information processing apparatus 1A obtains the review result RR from the device via the communication unit 23.

Examples of the language model LM include, but are not limited to, large language models (LLMs) such as Bidirectional Encoder Representations from Transformers (BERT), Generative Pre-trained Transformer (GPT), Text-to-Text Transfer Transformer (T5), Robustly optimized BERT approach (RoBERTa), and Efficiently Learning an Encoder that Classifies Token Replacements Accurately (ELECTRA), learning models (e.g., Chat Generative Pre-trained Transformer (ChatGPT)) generated by performing transfer learning or fine tuning using a pre-trained model, and the like.

Examples of the data stored in the storage unit 21 further include the written request WR, the prompt, and the review result RR not illustrated in FIG. 4.

### (Input/Output Unit 22)

The input/output unit 22 is an interface with an input device that receives a data input and an output device that outputs data. Examples of the input device include, but are not limited to, a microphone, a camera, a line-of-sight input device, a keyboard, and a touch pad. Examples of the output device include, but are not limited to, a speaker and a liquid crystal display.

### (Communication Unit 23)

The communication unit 23 is an interface for exchanging data via a network. Examples of the communication unit 23 include, but are not limited to, communication chips in various communication standards such as Ethernet (registered trademark), Wi-Fi (registered trademark), and wireless communication standards of mobile data communication networks, and connectors compliant with a universal serial bus (USB).

A specific configuration of the network is not particularly limited, but as an example, a wireless local area network (LAN), a wired LAN, a wide area network (WAN), a public line network, a mobile data communication network, or a combination of those networks may be used.

### (Control Unit 10)

The control unit 10 controls each component included in the information processing apparatus 1A. As illustrated in FIG. 4, the control unit 10 includes an acquisition unit 11, a generation unit 12, an extraction unit 13, and an output unit 14. In the present illustrative example embodiment, the acquisition unit 11, the generation unit 12, and the extraction unit 13 implement an acquisition means, a generation means, and an extraction means, respectively.

### (Acquisition Unit 11)

The acquisition unit 11 obtains data output from the input/output unit 22 or the communication unit 23. The acquisition unit 11 stores the obtained data in the storage unit 21. As an example, the acquisition unit 11 obtains the written request WR for requesting a change in the specifications of the electronic medical record.

### (Generation Unit 12)

The generation unit 12 generates the review result RR, which is a result of the review of the written request WR. As an example, the generation unit 12 inputs, to the language model LM, the written request WR and the prompt for instructing the execution of the review of the written request WR stored in the storage unit 21, thereby generating the review result RR of the written request WR. The generation unit 12 stores the generated review result RR in the storage unit 21.

As an example, the generation unit 12 may obtain a predetermined prompt from the storage unit 21, and may input the predetermined prompt to the language model LM. As another example, a prompt associated with a review item may be stored in the storage unit 21, and the generation unit 12 may input, for each review item, the prompt associated with the review item to the language model LM.

The generation unit 12 may generate the review result RR using Retrieval-Augmented Generation (RAG). For example, the generation unit 12 searches a knowledge base storing system-specific knowledge, information, and the like of electronic medical records in accordance with the written request WR. Then, the generation unit 12 inputs, to the language model LM, a search result, the written request WR, and the prompt, and obtains the review result RR from the language model LM.

The generation unit 12 may generate the review result RR by inputting, to the language model LM, review target information extracted from the written request WR by the extraction unit 13 to be described later and the prompt.

### (Extraction Unit 13)

The extraction unit 13 extracts the review target information, which is subject to the review, from the written request WR. The extraction unit 13 supplies the extracted review target information to the generation unit 12.

As an example, the extraction unit 13 extracts, as the review target information, a description of a predetermined item from the written request WR written in a predetermined format. For example, a case is assumed in which the following items are included in the written request WR.
“Background” item describing a background, a purpose, and the like for which a change in the specifications is requested
“Common” item describing check items for requesting a change in the specifications
“Functional specifications” item describing detailed changes

The extraction unit 13 extracts, as the review target information, the “background” item and the “functional specifications” item to be reviewed from among the items described above.

As another example, the extraction unit 13 carries out a search with predetermined text in the written request WR, and extracts the review target information based on a result of the search. For example, the extraction unit 13 searches the written request WR for the text such as “background”, “purpose”, and “request details”. Then, the extraction unit 13 extracts, as the review target information, text associated with the text found in the search.

In this manner, by the extraction unit 13 extracting the review target information, the load of the processing in the language model LM may be reduced.

### (Output Unit 14)

The output unit 14 outputs data to another device via the input/output unit 22 or the communication unit 23. As an example, the output unit 14 outputs the review result RR stored in the storage unit 21 to the another device via the communication unit 23.

### (Flow of Process Executed by Information Processing Apparatus 1A)

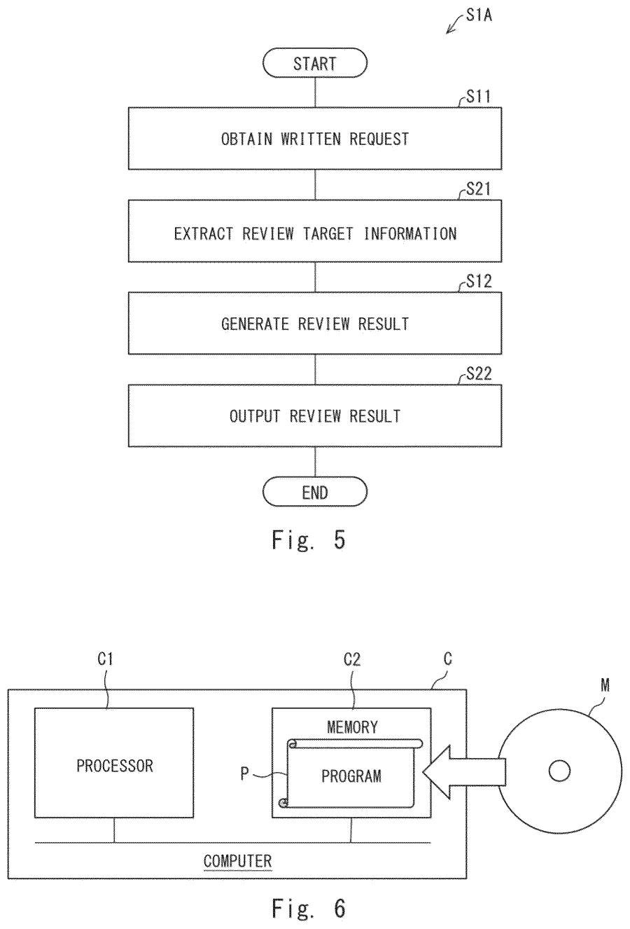

A flow of a process (information processing method S1A) to be executed by the information processing apparatus 1A will be described with reference to FIG. 5. FIG. 5 is a flowchart illustrating the flow of the information processing method S1A.

### (Step S11)

In step S11, the acquisition unit 11 obtains the written request WR for requesting a change in the specifications of the electronic medical record. The acquisition unit 11 stores the obtained written request WR in the storage unit 21.

### (Step S21)

In step S21, the extraction unit 13 extracts the review target information, which is subject to the review, from the written request WR stored in the storage unit 21. The extraction unit 13 supplies the extracted review target information to the generation unit 12.

### (Step S12)

In step S12, the generation unit 12 inputs the review target information extracted by the extraction unit 13 and a prompt for instructing execution of a review to the language model LM, thereby generating the review result RR of the written request WR. The generation unit 12 stores the review result RR in the storage unit 21.

If the extraction unit 13 fails to extract the review target information in step S21 (if step S21 is not executed), the generation unit 12 inputs the written request WR and the prompt for instructing the execution of the review to the language model LM, thereby generating the review result RR of the written request WR.

### (Step S22)

In step S22, the output unit 14 outputs the review result stored in the storage unit 21.

### (First Example of Review Result RR)

As mentioned above, the content to be reviewed by the information processing apparatus 1A is not particularly limited. As an example, the information processing apparatus 1A may review the format of the written request WR. As an example of this case, the information processing apparatus 1A may review, as a review item, whether any description is omitted, whether any prohibited character is used, whether any erroneous description is included, or the like.

For example, the generation unit 12 inputs, to the language model LM, the written request WR and the prompt “Review the format, and output a determination result and advice.”, and generates an output of the language model LM as the review result RR. For example, the generation unit 12 generates the review result RR including the following.
Review item “whether a background/reason is written”
Determination result “positive”
Advice list “The background and reason are clearly stated.”

With this configuration, the information processing apparatus 1A generates the review result of the format of the written request WR, whereby the creation of the written request WR may be supported.

### (Second Example of Review Result RR)

The information processing apparatus 1A may carry out a review specific to the written request WR of the electronic medical record. As an example of the review specific to the written request WR of the electronic medical record, the information processing apparatus 1A may review a patient information handover. As an example of this case, the information processing apparatus 1A may review, as a review item, whether an influence on a system of a section related to patient information, which is a system cooperating with the electronic medical record (which will also be referred to as a “subsystem” hereinafter), is taken into consideration according to a change in the specifications.

For example, the generation unit 12 inputs, to the language model LM, the written request WR and a prompt “Review whether presence or absence of a relevant subsystem is written, and output a determination result and advice.”, and generates an output of the language model LM as the review result RR. For example, the generation unit 12 generates the review result RR including the following.
Review item “whether presence or absence of a relevant subsystem is written”
Determination result “negative”
Advice list “No description regarding the subsystem relevant to input text is found, check and write the presence or absence of the relevant system.”

With this configuration, the information processing apparatus 1A generates the review result as to whether the patient information handover is considered in the written request WR, whereby the creation of the written request WR may be supported.

### (Third Example of Review Result RR)

As another example of the review specific to the written request WR of the electronic medical record, the information processing apparatus 1A may carry out a review regarding suppression of falsification of the electronic medical record information. As an example of this case, the information processing apparatus 1A may review, as a review item, whether an influence on a progress note (PN) is taken into consideration according to a change in the specifications.

For example, the generation unit 12 inputs, to the language model LM, the written request WR and a prompt “Review whether an influence on the PN is taken into consideration if a display item is added, and output a determination result and advice.”, and generates an output of the language model LM as the review result RR. For example, the generation unit 12 generates the review result RR including the following.
Review item “whether an influence on the PN is considered if a display item is added”
Determination result “negative”
Advice list “While there is no description regarding addition of display items, there is a description regarding information handover setting, and accordingly, add or modify the display items.”

With this configuration, the information processing apparatus 1A generates the review result as to whether the suppression of the falsification of the electronic medical record information is considered in the written request WR, whereby the creation of the written request WR may be supported.

### (Fourth Example of Review Result RR)

As still another example of the review specific to the written request WR of the electronic medical record, the information processing apparatus 1A may review authority according to a job category of the user of the electronic medical record. As an example of this case, the information processing apparatus 1A may review, as a review item, whether the authority depending on whether the electronic medical record is used by a doctor or a nurse is set.

For example, the generation unit 12 inputs, to the language model LM, the written request WR and a prompt “Review whether functional content is written if there is a difference in functions depending on whether the user of the electronic medical record is a doctor or a nurse, and output a determination result and advice.”, and generates an output of the language model LM as the review result RR. For example, the generation unit 12 generates the review result RR (review result RR of the review regarding the authority depending on whether the user of the electronic medical record is at least a doctor or a nurse) including the following.
Review item “whether functional content is written if there is a difference in functions depending on whether the user of the electronic medical record is a doctor or a nurse”
Determination result “negative”

Advice list “There is no description regarding whether there is a difference in functions depending on whether the user of the electronic medical record is a doctor or a nurse, describe what functions a doctor may use and what functions a nurse may use”.

The job category is not limited to the doctor and the nurse described above, and only needs to be a job category of the user who uses the electronic medical record. Examples of the job category may include a midwife, pharmacist, radiologist, physical therapist, occupational therapist, clinical engineer, registered dietitian, and doctor clerical work assistant.

With this configuration, the information processing apparatus 1A generates the review result as to whether the authority according to the job category of the user of the electronic medical record is considered in the written request WR, whereby the creation of the written request WR may be supported.

### (Fifth Example of Review Result RR)

As yet another example of the review specific to the written request WR of the electronic medical record, the information processing apparatus 1A may review linkage between the electronic medical record and the cooperative subsystem. As an example of this case, the information processing apparatus 1A may review, as a review item, whether the operation of the subsystem in a case where, in the electronic medical record, the operation is stopped or a displayed image is closed is taken into consideration.

For example, the generation unit 12 inputs, to the language model LM, the written request WR and a prompt “Review whether the operation upon a ”cancel“ or ”close“ button is pressed is taken into consideration, and output a determination result and advice.”, and generates an output of the language model LM as the review result RR. For example, the generation unit 12 generates the review result RR (review result RR of the review regarding the linkage between the electronic medical record and the cooperative subsystem) including the following.
Review item “whether the operation upon the “cancel” or “close” button is pressed is considered”
Determination result “negative”
Advice list “Since there is no description regarding the operation upon the “cancel” or “close” button is pressed, add a description to the functional specifications in consideration of this point.”

With this configuration, the information processing apparatus 1A generates the review result as to whether the linkage between the electronic medical record and the subsystem is considered in the written request WR, whereby the creation of the written request WR may be supported.

### (Exemplary Process Performed by Generation Unit 12)

As described in the examples of the review result RR described above, the generation unit 12 may generate the review result RR for each review item. Meanwhile, the generation unit 12 may generate the review result RR of a plurality of review items.

For example, in a case where the language model LM has trained to review a plurality of review items (e.g., equal to or more than two review items among the review items of the first to fifth examples of the review result RR described above), the generation unit 12 inputs the written request WR (or the review target information) and a prompt “Review and output a determination result and advice.” to the language model LM. The generation unit 12 generates the output of the language model LM as the review result RR. As an example, the generation unit 12 generates the review result RR of at least one of the review regarding the patient information handover and the review regarding the suppression of the falsification of the electronic medical record information.

With this configuration, the information processing apparatus 1A collectively obtains the review results RR of the plurality of review items from the language model LM, whereby the processing may be reduced.

### (Effects of Information Processing Apparatus 1A)

As described above, the information processing apparatus 1A inputs, to the language model LM, the written request WR (or the review target information) and the prompt for instructing the execution of the review of the written request WR, thereby generating the review result RR of the written request WR.

Thus, according to the information processing apparatus 1A, it becomes possible to generate the review result RR obtained by reviewing whether the written request WR is inappropriate in terms of the format of the written request WR and the content specific to the electronic medical record.

The information processing apparatus 1A outputs the review result RR. Thus, the information processing apparatus 1A may notify a creator of the written request WR of an inappropriate point of the written request WR.

Thus, according to the information processing apparatus 1A, it becomes possible to support the creation of the written request WR for requesting a change in the specifications of the electronic medical record.

### Exemplary Implementation by Software

Some or all of the functions of the information processing apparatuses 1 and 1A (which will also be referred to as “each of the above apparatuses” hereinafter) may be implemented by hardware such as an integrated circuit (IC chip), or may be implemented by software.

In the latter case, each of the above apparatuses is implemented by, for example, a computer that executes commands of a program, which is software for implementing each function. An example of such a computer (which will be referred to as a computer C hereinafter) is illustrated in FIG. 6. FIG. 6 is a block diagram illustrating a hardware configuration of the computer C that functions as each of the above apparatuses.

The computer C includes at least one processor C1 and at least one memory C2. A program P for causing the computer C to operate as each of the above apparatuses is recorded in the memory C2. In the computer C, the processor C1 reads the program P from the memory C2 and executes it, thereby implementing the functions of each of the above apparatuses.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination thereof may be used. As the memory C2, for example, a flash memory, a hard disk drive (HDD), a solid state drive (SSD), or a combination thereof may be used.

The computer C may further include a random access memory (RAM) for loading the program P at the time of execution and temporarily storing various types of data. The computer C may further include a communication interface for exchanging data with another device. The computer C may further include an input/output interface for connecting input/output devices, such as a keyboard, a mouse, a display, a printer, and the like.

The program P may be recorded in a non-transitory tangible recording medium M readable by the computer C. As such a recording medium M, for example, a tape, a disk, a card, a semiconductor memory, or a programmable logic circuit may be used. The computer C may obtain the program P via such a recording medium M. The program P may be transmitted via a transmission medium. As such a transmission medium, for example, a communication network or a broadcast wave may be used. The computer C may also obtain the program P via such a transmission medium.

The functions of each of the above apparatuses may be implemented by a single processor provided in a single computer, may be implemented in cooperation with a plurality of processors provided in a single computer, or may be implemented in cooperation with a plurality of processors provided in each of a plurality of computers.

The program for causing each of the above apparatuses to implement the functions described above may be stored in a single memory provided in a single computer, may be stored in a distributed manner in a plurality of memories provided in a single computer, or may be stored in a distributed manner in a plurality of memories provided in each of a plurality of computers.

Each of the drawings is merely an example for describing one or more example embodiments. Each of the drawings is not associated with only one specific example embodiment, but may be associated with one or more other example embodiments. As will be appreciated by those of ordinary skill in the art, various features or steps described with reference to any one of the drawings may be combined with features or steps illustrated in one or more other drawings, for example, to create an example embodiment not explicitly illustrated or described. All of the features or steps illustrated in any one of the drawings for describing illustrative example embodiments are not necessarily mandatory, and some features or steps may be omitted. The order of the steps described in any of the drawings may be changed as appropriate.

### Supplementary Information A

The present disclosure includes techniques described in the following Supplementary Notes. However, the present disclosure is not limited to the techniques described in the following Supplementary Notes, and various modifications may be made within the scope described in the claims.

### (Supplementary Note A1)

An information processing apparatus including:
an acquisition means for obtaining a written request for requesting a change in a specification of an electronic medical record; and
a generation means for generating a review result of the written request by inputting the written request and a prompt for instructing execution of a review of the written request to a language model that has been subjected to machine learning,
in which the generation means generates the review result of at least one of the review regarding a patient information handover or the review regarding suppression of falsification of information associated with the electronic medical record.

### (Supplementary Note A2)

The information processing apparatus according to Supplementary Note A1, further including an extraction means for extracting review target information, which is subject to the review, from the written request,
in which the generation means generates the review result of the written request by inputting the review target information and the prompt to the language model.

### (Supplementary Note A3)

The information processing apparatus according to Supplementary Note A1 or A2, in which the generation means generates the review result of the review regarding authority depending on whether a user of the electronic medical record is at least a doctor or a nurse.

### (Supplementary Note A4)

The information processing apparatus according to any one of Supplementary Notes A1 to A3, in which the generation means generates the review result of the review regarding linkage between the electronic medical record and a cooperative subsystem.

### Supplementary Information B

The present disclosure includes techniques described in the following Supplementary Notes. However, the present disclosure is not limited to the techniques described in the following Supplementary Notes, and various modifications may be made within the scope described in the claims.

### (Supplementary Note B1)

An information processing method including:
acquisition processing in which at least one processor obtains a written request for requesting a change in a specification of an electronic medical record; and
generation processing in which the at least one processor generates a review result of the written request by inputting the written request and a prompt for instructing execution of a review of the written request to a language model that has been subjected to machine learning,
in which the at least one processor generates, in the generation processing, the review result of at least one of the review regarding a patient information handover or the review regarding suppression of falsification of information associated with the electronic medical record.

### (Supplementary Note B2)

The information processing method according to Supplementary Note B1, further including extraction processing in which the at least one processor extracts review target information, which is subject to the review, from the written request,
in which the at least one processor generates, in the generation processing, the review result of the written request by inputting the review target information and the prompt to the language model.

### (Supplementary Note B3)

The information processing method according to Supplementary Note B1 or B2, in which the at least one processor generates, in the generation processing, the review result of the review regarding authority depending on whether a user of the electronic medical record is at least a doctor or a nurse.

### (Supplementary Note B4)

The information processing method according to any one of Supplementary Notes B1 to B3, in which the at least one processor generates, in the generation processing, the review result of the review regarding linkage between the electronic medical record and a cooperative subsystem.

### Supplementary Information C

The present disclosure includes techniques described in the following Supplementary Notes. However, the present disclosure is not limited to the techniques described in the following Supplementary Notes, and various modifications may be made within the scope described in the claims.

### (Supplementary Note C1)

An information processing program for causing a computer to function as an information processing apparatus, the program causing the computer to function as means including:
an acquisition means for obtaining a written request for requesting a change in a specification of an electronic medical record; and
a generation means for generating a review result of the written request by inputting the written request and a prompt for instructing execution of a review of the written request to a language model that has been subjected to machine learning,
in which the generation means generates the review result of at least one of the review regarding a patient information handover or the review regarding suppression of falsification of information associated with the electronic medical record.

### (Supplementary Note C2)

The information processing program according to Supplementary Note C1, the program causing the computer to function as the means further including an extraction means for extracting review target information, which is subject to the review, from the written request,
in which the generation means generates the review result of the written request by inputting the review target information and the prompt to the language model.

### (Supplementary Note C3)

The information processing program according to Supplementary Note C1 or C2, in which the generation means generates the review result of the review regarding authority depending on whether a user of the electronic medical record is at least a doctor or a nurse.

### (Supplementary Note C4)

The information processing program according to any one of Supplementary Notes C1 to C3, in which the generation means generates the review result of the review regarding linkage between the electronic medical record and a cooperative subsystem.

### Supplementary Information D

The present disclosure includes techniques described in the following Supplementary Notes. However, the present disclosure is not limited to the techniques described in the following Supplementary Notes, and various modifications may be made within the scope described in the claims.

### (Supplementary Note D1)

An information processing apparatus including:
at least one processor,
in which the at least one processor is configured to perform:
acquisition processing that obtains a written request for requesting a change in a specification of an electronic medical record; and
generation processing that generates a review result of the written request by inputting the written request and a prompt for instructing execution of a review of the written request to a language model that has been subjected to machine learning, and
in the generation processing, the at least one processor generates the review result of at least one of the review regarding a patient information handover or the review regarding suppression of falsification of information associated with the electronic medical record.

The information processing apparatus may further include a memory. The memory may store a program for causing the at least one processor to execute each of the processing.

### (Supplementary Note D2)

The information processing apparatus according to Supplementary Note D1, in which
the at least one processor further performs extraction processing that extracts review target information, which is subject to the review, from the written request, and
in the generation processing, the at least one processor generates the review result of the written request by inputting the review target information and the prompt to the language model.

### (Supplementary Note D3)

The information processing apparatus according to Supplementary Note D1 or D2, in which the at least one processor generates, in the generation processing, the review result of the review regarding authority depending on whether a user of the electronic medical record is at least a doctor or a nurse.

### (Supplementary Note D4)

The information processing apparatus according to any one of Supplementary Notes D1 to D3, in which the at least one processor generates, in the generation processing, the review result of the review regarding linkage between the electronic medical record and a cooperative subsystem.

### Supplementary Information E

The present disclosure includes techniques described in the following Supplementary Notes. However, the present disclosure is not limited to the techniques described in the following Supplementary Notes, and various modifications may be made within the scope described in the claims.

### (Supplementary Note E1)

A non-transitory recording medium recording an information processing program for causing a computer to function as an information processing apparatus, the information processing program causing the computer to perform:
acquisition processing that obtains a written request for requesting a change in a specification of an electronic medical record; and
generation processing that generates a review result of the written request by inputting the written request and a prompt for instructing execution of a review of the written request to a language model that has been subjected to machine learning,
in which the computer generates, in the generation processing, the review result of at least one of the review regarding a patient information handover or the review regarding suppression of falsification of information associated with the electronic medical record.

What is claimed is:

1. An information processing apparatus comprising:
at least one memory storing instructions; and
at least one processor configured to execute the instructions to:
obtain a written request for requesting a change in a specification of an electronic medical record; and
generate a review result of the written request by inputting the written request and a prompt for instructing execution of a review of the written request to a language model that has been subjected to machine learning,
wherein the at least one processor is configured to execute the instructions to generate the review result of at least one of the review regarding a patient information handover or the review regarding suppression of falsification of information associated with the electronic medical record.

2. The information processing apparatus according to claim 1, wherein
the at least one processor is configured to execute the instructions to:
extract review target information, which is subject to the review, from the written request; and
generate the review result of the written request by inputting the review target information and the prompt to the language model.

3. The information processing apparatus according to claim 1, wherein the at least one processor is configured to execute the instructions to generate the review result of the review regarding authority depending on whether a user of the electronic medical record is at least a doctor or a nurse.

4. The information processing apparatus according to claim 1, wherein the at least one processor is configured to execute the instructions to generate the review result of the review regarding linkage between the electronic medical record and a cooperative subsystem.

5. An information processing method comprising:
obtaining a written request for requesting a change in a specification of an electronic medical record; and
generating a review result of the written request by inputting the written request and a prompt for instructing execution of a review of the written request to a language model that has been subjected to machine learning,
wherein the generating the review result includes generating the review result of at least one of the review regarding a patient information handover or the review regarding suppression of falsification of information associated with the electronic medical record.

6. A non-transitory computer-readable medium storing an information processing program for causing a computer to function as an information processing apparatus, the information processing program causing the computer to perform a process comprising:
obtaining a written request for requesting a change in a specification of an electronic medical record; and
generating a review result of the written request by inputting the written request and a prompt for instructing execution of a review of the written request to a language model that has been subjected to machine learning,
wherein the generating the review result includes generating the review result of at least one of the review regarding a patient information handover or the review regarding suppression of falsification of information associated with the electronic medical record.
